(19) United States
(12) Patent Application Publication
(10) Pub. No.: US 2009/0043080 A1
(43) Pub. Date: Feb. 12, 2009

(54) Purification of a Bulk of a Factor VII Polypeptide by Fractionated Elution from an Anion-Exchange Material

(75) Inventors: Uffe Clausen, Kalundborg (DK); Christian Fangel, Kalundborg (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK HEALTHCARE A/G, Zurich (CH)

(21) Appl. No.: 11/663,389

(22) PCT Filed: Sep. 29, 2005
(86) PCT No.: PCT/EP05/54906

§ 371 Date: Oct. 24, 2008

(30) Foreign Application Priority Data
Sep. 29, 2004 (DK) PA 2004 01478
Sep. 29, 2004 (DK) PA 2004 01480

(51) Int. Cl. C07K 1/14

(52) U.S. Cl. 530/416

(57) ABSTRACT

The present invention relates to the purification of a Factor VII polypeptide from a bulk of a Factor VII polypeptide with respect to desirable glycoforms by fractionated elution from an anion-exchange material with an eluting buffer comprising a certain concentration of calcium ions. The present invention renders it possible to enrich a bulk of a Factor VII polypeptide with respect to desirable glycoforms.

### FIELD OF THE INVENTION

The present invention relates to the purification of a Factor VII polypeptide from a bulk of a Factor VII polypeptide with respect to desirable glycoforms by fractionated elution from an anion-exchange material with an eluting buffer comprising a certain concentration of calcium ions. The present invention renders it possible to enrich a bulk of a Factor VII polypeptide with respect to desirable glycoforms. The present invention also renders it possible to utilize bulks of a Factor VII polypeptide which have a relatively low abundance of desirable glycoforms of a Factor VII polypeptide.

### BACKGROUND OF THE INVENTION

The proteins involved in the clotting cascade, including, e.g., Factor VII, Factor VIII, Factor IX, Factor X, and Protein C, are proving to be useful therapeutic agents to treat a variety of pathological conditions. Accordingly, there is an increasing need for formulations comprising these proteins that are pharmaceutically acceptable and exhibit a uniform and predetermined clinical efficacy.

Because of the many disadvantages of using human plasma as a source of pharmaceutical products, it is preferred to produce these proteins in recombinant systems. The clotting proteins, however, are subject to a variety of co- and posttranslational modifications, including, e.g., asparagine-linked (N-linked) glycosylation; O-linked glycosylation; and γ-carboxylation of Glu residues. These modifications may be qualitatively or quantitatively different when heterologous cells are used as hosts for large-scale production of the proteins. In particular, production in heterologous cells often results in a different array of glycoforms that represent identical polypeptides having different covalently linked oligosaccharide structures.

In different systems, variations in the oligosaccharide structure of therapeutic proteins have been linked to, i.a., changes in immunogenicity and in vivo clearance. Thus, there is a need in the art for commercial Factor VII polypeptide compositions that contain predetermined glycoform patterns (or at least being enriched with respect to certain desirable glycoforms), in particular a purified bulk of a Factor VII polypeptide that has a desirably high content of sialylated Factor VII polypeptide structures.

### SUMMARY OF THE INVENTION

The present invention solves the problem of providing a purified bulk of a Factor VII polypeptide that has a desirably high content of sialylated Factor VII polypeptide structures by utilizing a fractionated elution of a bulk of a Factor VII polypeptide from an anion-exchange material.

Thus, a first aspect of the present invention relates to an industrial-scale process for the purification of a bulk of a Factor VII polypeptide, said process comprising the step of:

(a) contacting the bulk of the Factor VII polypeptide with an anion-exchange material under conditions which facilitate binding of a portion of said bulk of the Factor VII polypeptide to said anion-exchange material;

(b) eluting said anion-exchange material with a first elution buffer comprising Ca²⁺ in a concentration of up to a threshold value of X mM; and

(c) eluting said anion-exchange material with a second elution buffer comprising Ca²⁺ in a concentration of more than the threshold value of X mM, and collecting a purified bulk of the Factor VII polypeptide as an eluate;

wherein the threshold value, X, is selected so that at least 5% by weight of the bulk of the Factor VII polypeptide is removed from said anion-exchange material in the first elution step (b), and at least 50% by weight of the bulk of the Factor VII polypeptide can be collected as the purified bulk of the Factor VII polypeptide in the second elution step (c).

In a second aspect of the present invention, the threshold value is—in absolute terms—in the range of 3-12 mM, e.g. 4-11 mM, such as 5-10 mM.

The novel industrial-scale processes render it possible to purify a crude bulk of a Factor VII polypeptide with respect to desirable glycoforms, in particular—but not exclusively—crude bulks having a fairly low abundance of desirable glycoforms of Factor VII polypeptides.

Thus, a third aspect of the present invention relates to an industrial scale process for the production and purification of a bulk of a Factor VII polypeptide, said process including the steps of

(i) producing a crude bulk of the Factor VII polypeptide in a cell culture, and

(ii) purifying said crude bulk of the Factor VII polypeptide by a purification sequence utilizing one or more anion-exchange purification processes,

wherein at least one of such anion-exchange purification processes is conducted as defined hereinabove.

### DETAILED DESCRIPTION OF THE INVENTION

As mentioned above, the present invention provides an industrial-scale process for the purification of a bulk of a Factor VII polypeptide, said process comprising the step of:

(a) contacting the bulk of the Factor VII polypeptide with an anion-exchange material under conditions which facilitate binding of a portion of said bulk of the Factor VII polypeptide to said anion-exchange material;

(b) eluting said anion-exchange material with a first elution buffer comprising Ca²⁺ in a concentration of up to a threshold value of X mM; and

(c) eluting said anion-exchange material with a second elution buffer comprising Ca²⁺ in a concentration of more than the threshold value of X mM, and collecting a purified bulk of the Factor VII polypeptide as an eluate;

wherein the threshold value, X, is selected so that at least 5% by weight of the bulk of the Factor VII polypeptide is removed from said anion-exchange material in the first elution step (b), and at least 50% by weight of the bulk of the Factor VII polypeptide can be collected as the purified bulk of the Factor VII polypeptide in the second elution step (c).

In an alternative variant of the process of the present invention, the threshold value is defined in absolute terms, i.e. the present invention also provides an industrial-scale process for the purification of a bulk of a Factor VII polypeptide, said process comprising the step of:

(a) contacting the bulk of the Factor VII polypeptide with an anion-exchange material under conditions which facilitate binding of a portion of said bulk of the Factor VII polypeptide to said anion-exchange material;

(b) eluting said anion-exchange material with a first elution buffer comprising Ca²⁺ in a concentration of up to a threshold value of X mM; and

(c) eluting said anion-exchange material with a second elution buffer comprising Ca²⁺ in a concentration of more than the threshold value of X mM, and collecting a purified bulk of the Factor VII polypeptide as an eluate;

wherein the threshold value, X, is in the range of 3-12 mM.

The processes of the present invention are particularly feasible for “industrial-scale” (or “large-scale”) bulks of a Factor VII polypeptide. By the term “industrial-scale” is typically meant methods wherein the volume of liquid Factor VII polypeptide compositions is at least 100 L, such as at least 500 L, e.g. at least 1000 L, or at least 5000 L, or where the weight of the compositions is at least 100 kg, such as at least 500 kg, e.g. at least 1000 kg, or at least 5000 kg, or where the weight of the product is at least 1 g (dry matter), such as at least 10 g, e.g. at least 50 g, e.g. 1-1000 g or 1-500 g or 1-100 g.

In the present context, the terms “glycoform” and “glycoforms” refer to form(s) of an otherwise sequence-identical Factor VII polypeptide having different covalently linked oligosaccharide structures, in particular with respect to the presence or absence of sialyl groups.

In the present context, the term “purification” means removal of undesirable glycoforms of the Factor VII polypeptide, i.e. glycoforms which has an incomplete sialyl pattern (e.g. no or an incomplete number of sialyl groups).

It has been found that such undesirable glycoforms (with an incomplete sialyl pattern) elutes before “desirable glycoforms” (i.e. glycoforms with a “complete” sialyl pattern) when using a calcium (Ca²⁺)-containing buffer(s) in combination with anion-exchange chromatography. Thus, by the fractionated elution defined herein, it is possible to obtain a purified bulk of a Factor VII polypeptide enriched with respect to a desirable glycoform or desirable glycoforms.

The expression “bulk” used herein is intended to mean a solid mass as well as a liquid mass, e.g. a solution or suspension comprising the Factor VII polypeptide. The expression “bulk” is in particular meant to refer to a “large” volume or mass, i.e. referring to volumes and masses known from large-scale and industrial-scale processes.

The term “Factor VII polypeptide” is defined further below.

The “threshold value” is of utmost importance in the present context as it defines an important parameter for the fractionated elution using Ca²⁺ elution buffers. The threshold value defines the borderline between the fractions being collected to form the purified bulk of the Factor VII polypeptide, and the preceding fractions being discarded, processed or recycled/reused. For an anion-exchange material arranged in a column (most usual and useful arrangement), the threshold value corresponds to the concentration of Ca²⁺ in the buffer at the inlet of the column, and it should be understood that the corresponding eluate is collected with a delay in time corresponding to the time it takes for the “front” of the eluting buffer to pass through the column.

Step (a)—Contacting the Bulk with an Anion-Exchange Material

In a first step of the process, the bulk of the Factor VII polypeptide is contacted with an anion-exchange material. The aim is to facilitate binding of a portion of said bulk of the Factor VII polypeptide to said anion-exchange material.

By the term “portion” in connection with step (a) is meant at least 30% (i.e. 30-100%) of the mass of the Factor VII polypeptide present in the bulk of the Factor VII polypeptide. It should be understood that it in most instances is desirable to bind far more than 30% of the mass of the Factor VII polypeptides, e.g. at least 50%, or at least 70%, or a predominant portion. By the term “predominant portion” is meant at least 90% of the mass of the Factor VII polypeptide present in the bulk of the Factor VII polypeptide. Preferably an even higher portion becomes bound to the anion-exchange material, e.g. at least 95% of the mass, or at least 98% of the mass, or at least 99% of the mass, or even substantially all of the mass of the Factor VII polypeptide present in the bulk of the Factor VII polypeptide.

The bulk of the Factor VII polypeptide typically originates from an industrial-scale production process, e.g. a cell culture, a cloned animal (e.g. cows, pigs, sheep, goats, and fish) or insect, or the like, in particular from a cell culture.

The anion-exchange material is preferably a strong anion-exchange material, e.g. an anion-exchange material having quaternary ammonium groups. Commercial examples of such materials are Q-Sepharose Fast Flow from Amersham Biosciences and POROS HQ 50 from Tosohaas.

The most common arrangement of the anion-exchange material is in the format of a column. Arrangement in a batch container is of course also possible.

The bulk of the Factor VII polypeptide is typically obtained directly from a preceding purification step, or from a preceding purification step with subsequent adjustment of pH, ionic strength, etc., whatever necessary.

Typically, the pH of the bulk of the Factor VII polypeptide bulk is in the range of 7.5-9.5, such as 8.0-9.0, and the conductivity is typically in the range of 5-30 mS/cm, such as 10-20 mS/cm. The temperature of the bulk is typically, but without limitation, 0-15° C., such as around 2-10° C.

The contacting of the bulk of the Factor VII polypeptide is typically conducted according to conventional protocols, i.e. the concentration, temperature, pH, ionic strength, etc. of the bulk may be as usual, and the anion-exchange material may be washed and equilibrated as usual.

The load of Factor VII polypeptide is typically in the range of 10-40 g, e.g. 15-30 g, Factor VII polypeptide per litre of matrix (anion-exchange material in wet form), and the bulk is typically applied at a flow of 3-200 column volumes per hour (CV/h), such as at least 10 CV/h, e.g. at least 20 CV/h or at least 40 CV/h or at least 80 CV/h, e.g. 80-120 CV/h.

After contacting and binding of the Factor VII polypeptide to the anion-exchange material, one or more washing step may be conducted before the elution steps (b) and (c).

Step (b)—First Elution Step

After binding of the bulk of the Factor VII polypeptide to the anion-exchange materials, a first elution step (b) is conducted in order to remove a fraction of the bulk of the Factor VII polypeptide, where said removed fraction has a lower content of sialylated Factor VII polypeptide structures than the original bulk. By this step, the remaining (bound) fraction of the Factor VII polypeptide on the anion-exchange material will have a higher content of sialylated Factor VII polypeptide structures than the original bulk.

The elution step (b) is conducted by using a first elution buffer comprising Ca²⁺ in a concentration of up to a threshold value of X mM.

The first elution buffer comprises Ca²⁺ (e.g. as calcium chloride) and a buffering agent, possibly in combination with other salt(s) (e.g. sodium salts, potassium salts, and magnesium salts, such as sodium chloride, potassium chloride, magnesium chloride, magnesium acetate, magnesium gluconate, and magnesium laevulate). The buffering agent is typically at least one component selected from the groups consisting of acids and salts of MES, PIPES, ACES, BES, TES, HEPES, TRIS, histidine, imidazole, glycine, glycylglycine, glycinamide, phosphoric acid, acetic acid (e.g. sodium acetate), lactic acid, glutaric acid, citric acid, tartaric acid, malic acid, maleic acid, and succinic acid. It should be understood that the buffering agent may comprise a mixture of two or more components, wherein the mixture is able to provide a pH value in the specified range. As examples can be mentioned acetic acid and sodium acetate, etc.

The crucial threshold concentration of Ca²⁺, X, is selected so that at least 5% by weight, such as at least 10% by weight, or at least 15% by weight, of the bulk of the Factor VII polypeptide is removed from said anion-exchange material in the first elution step (b) (see also further below). The fraction thus removed represents a fairly high proportion of the undesirable glycoform.

In a preferred embodiment, the threshold value, X, is in the range of 3-12 mM, e.g. 4-11 mM, such as 5-10 mM.

The temperature of the anion-exchange material with the bound Factor VII polypeptide is typically 0-15° C., such as around 2-10° C., e.g. kept within a specified range by using a cooling jacket.

It should be understood that the concentration of Ca²⁺ in the first elution buffer must be more than 0 (zero), e.g. at least 1 mM, in order for the undesirable glycoforms to be removed by the first elution step (b). When a gradient buffer is used, the average concentration of Ca²⁺ in the first elution buffer must be more than 0 (zero), e.g. at least 1 mM.

In one embodiment, the first elution buffer is a batch of a buffer with a (constant) Ca²⁺ concentration in the range of 1-8 mM, such as in the range of 2-7 mM.

In a further preferred embodiment, the first elution step (b) is conducted by using a gradient buffer, in particular a gradient buffer having a final Ca²⁺ concentration corresponding to X. The initial Ca²⁺ concentration is typically in the range of 0-8 mM, such as 0-5 mM.

Step (c)—Second Elution Step

After the first elution step (b), the anion-exchange material is eluted with a second elution buffer comprising Ca²⁺ in a concentration of more than the threshold value of X mM, and a purified bulk of the Factor VII polypeptide enriched with respect to desirable glycoforms of the Factor VII polypeptide can be collected as an eluate.

The second elution buffer comprises Ca²⁺ (although in a higher concentration than the first elution buffer) and a buffering agent, possibly in combination with other salt(s) (e.g. sodium salts, potassium salts, and magnesium salts, such as sodium chloride, potassium chloride, magnesium chloride, magnesium acetate, magnesium gluconate, and magnesium laevulate). The buffering agent is typically selected as specified above for the first elution buffer.

The crucial threshold concentration of Ca²⁺, X, is selected so that at least 50% by weight, such at least 60% by weight, or 70% by weight, of the bulk of the Factor VII polypeptide can be collected as the purified bulk of the Factor VII polypeptide in the second elution step (c).

As above, the threshold value, X, is preferably in the range of 3-12 mM, e.g. 4-11 mM, such as 5-10 mM.

In one embodiment, the second elution buffer is a batch of a buffer with a (constant) Ca²⁺ concentration in the range of 8-25 mM, such as in the range of 9-20 mM.

In one variant hereof, the first elution step (b) is conducted by using a first elution buffer in the form of a batch of a buffer with a (constant) Ca²⁺ concentration in the range of 1-8 mM, such as, e.g. in the range of 1-7 mM, 2-7 mM or 2-8 mM, and the second elution step (c) is conducted by using a second elution buffer in the form of a batch of a buffer with a (constant) Ca²⁺ concentration in the range of 8-25 mM, such as, e.g. in the range of 9-25 mM, 8-20 mM, or 9-20 mM.

In a further preferred embodiment, the second elution step (c) is conducted by using a gradient buffer, in particular a gradient buffer having an initial Ca²⁺ concentration corresponding to just above X. The final Ca²⁺ concentration is typically in the range of 10-25 mM, such as 12-20 mM.

In one very interesting embodiment, the first elution step (b) and the second elution step (c) are both conducted using a gradient buffer, such as a continuous gradient buffer. In one embodiment, the initial concentration of Ca²⁺ is 0-5 mM, such as in the range of 0-3 mM, e.g. about 0 mM, and a final concentration of Ca²⁺ of in the range of 10-25 mM, such as in the range of 12-20 mM, e.g. about 15 mM. As above, the threshold value, X, is typically in the range of 3-12 mM, e.g. 4-11 mM, such as 5-10 mM.

The overall elution process (step (b) and step (c)) is typically conducted at a flow of 3-200 column volumes per hour (CV/h), such as at least 10 CV/h, e.g. at least 20 CV/h or at least 40 CV/h or at least 80 CV/h, e.g. 20-120 CV/h, 20-80 CV/h, 20-60 CV/h, or 80-120 CV/h. Due to the fairly limited period of time at which the Factor VII polypeptide is present in a solution with a calcium concentration in an intermediate range, the risk of degradation of the product is minimized. Thus, the present inventors have not identified any stability problems in connection with the processes defined herein.

Another feature of the second elution step (c) is that the Factor VII polypeptide in many instances will be completely activated under the elution conditions. This is advantageous in that a separate activation step becomes unnecessary.

The term “purified bulk” means that the resulting bulk, i.e. the bulk collected in step (c), has a lower content of undesirable glycoforms of Factor VII polypeptide than the bulk applied in step (a). The term “purification” refers to the process wherein a purified bulk can be obtained, i.e. the process of the present invention.

Usually, the anion-exchange material is regenerated for the purpose of subsequent use by a sequence of steps.

Industrial-Scale Production and Purification

The present invention is particularly useful for industrial-scale production and purification of bulks of a Factor VII polypeptide. In such processes, the Factor VII polypeptide is typically produced by means of a cell culture.

Thus, the present invention also provides an industrial scale process for the production and purification of a bulk of a Factor VII polypeptide, said process including the steps of

(i) producing a crude bulk of the Factor VII polypeptide in a cell culture, and

(ii) purifying said crude bulk of the Factor VII polypeptide by a purification sequence utilizing one or more anion-exchange purification processes,

wherein at least one of such anion-exchange purification processes is conducted as defined hereinabove.

Preferably, the anion-exchange purification process as defined hereinabove is the last of the one or more anion-exchange purification processes.

One intriguing possibility is that the production step (i) needs not to be optimized with respect to the desirable glycoforms of the Factor VII polypeptide. Thus, in one interesting embodiment, the production step (i) is optimized with respect to the mass yield of the crude bulk of the Factor VII polypeptide. In such instances, the content of the desirable glycoform of the Factor VII polypeptide may be 80% or lower, and such “mass-optimized” bulks have hitherto had limited use for industrial production of Factor VII polypeptide products due to the difficulty in separating the undesirable glycoforms. The present invention, however, also provides an elegant solution to this problem.

Thus, in one variant thereof, mass content of sialylated Factor VII polypeptide structures in the crude bulk of the Factor VII polypeptide is at the most 80%, and the mass content of sialylated Factor VII polypeptide structures in the purified bulk of the Factor VII polypeptide is at least 90%.

Thus, in another variant thereof, mass content of sialylated Factor VII polypeptide structures in the crude bulk of the Factor VII polypeptide is at the most 90%, and the mass content of sialylated Factor VII polypeptide structures in the purified bulk of the Factor VII polypeptide is at least 95%.

Thus, in a still further variant thereof, mass content of sialylated Factor VII polypeptide structures in the crude bulk of the Factor VII polypeptide is at the most 93%, and the mass content of sialylated Factor VII polypeptide structures in the purified bulk of the Factor VII polypeptide is at least 96%.

In another variant thereof, the production of the crude bulk in the cell culture is conducted at a pH value of in the range of 7.0-7.6.

Typically, the production of the crude bulk is effected in a host cell, such as a eukaryotic host cell, e.g. a mammalian cell. In one embodiment of the invention, the mammalian cell is selected from the group consisting of HEK cells, BHK cells, CHO cells, COS cells, and myeloma cells such as SP2-0.

This being said, the production and purification (except for the fractionated elution purification step of the present invention) may be conducted as known to the person skilled in the art. Thus, industrial-scale production of a crude bulk of a Factor VII polypeptide can be conducted as disclosed in the present applicant's earlier applications, e.g. WO 04/027072 A2, WO 02/29083 A2, WO 02/29025 A2, WO 00/28065 A1, WO 02/77218 A1, etc.

Factor VII Polypeptide

As used herein, the term “Factor VII polypeptide” encompasses wild-type Factor VII (i.e. a polypeptide having the amino acid sequence disclosed in U.S. Pat. No. 4,784,950), as well as variants of Factor VII exhibiting substantially the same or improved biological activity relative to wild-type Factor VII. The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa. The term “Factor VII polypeptide” also encompasses polypeptides, including variants, in which the Factor VIIa biological activity has been substantially modified or somewhat reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

The biological activity of Factor VIIa in blood clotting derives from its ability to (i) bind to Tissue Factor (TF) and (ii) catalyze the proteolytic cleavage of Factor IX or Factor X to produce activated Factor IX or X (Factor IXa or Xa, respectively).

For the purposes of the invention, biological activity of Factor VII polypeptides (“Factor VII biological activity”) may be quantified by measuring the ability of a preparation to promote blood clotting, cf. Assay 4 described herein. In this assay, biological activity is expressed as the reduction in clotting time relative to a control sample and is converted to “Factor VII units” by comparison with a pooled human serum standard containing 1 unit/mL Factor VII activity. Alternatively, Factor VIIa biological activity may be quantified by (i) measuring the ability of Factor VIIa or a Factor VII-related polypeptide to produce activated Factor X (Factor Xa) in a system comprising TF embedded in a lipid membrane and Factor X. (Persson et al., J. Biol. Chem. 272:19919-19924, 1997); (ii) measuring Factor X hydrolysis in an aqueous system (“In Vitro Proteolysis Assay”, see Assay 2 below); (iii) measuring the physical binding of Factor VIIa or a Factor VII-related polypeptide to TF using an instrument based on surface plasmon resonance (Persson, FEBS Letts. 413:359-363, 1997); (iv) measuring hydrolysis of a synthetic substrate by Factor VIIa and/or a Factor VII-related polypeptide (“In Vitro Hydrolysis Assay”, see Assay 1 below); or (v) measuring generation of thrombin in a TF-independent in vitro system (see Assay 3 below).

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay (Assay 4), proteolysis assay (Assay 2), or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor VIIa that has been produced in the same cell type when tested in one or more of a clotting assay (Assay 4), proteolysis assay (Assay 2), or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, polypeptides having an amino acid sequence that differs from the sequence of wild-type Factor VII by insertion, deletion, or substitution of one or more amino acids.

Non-limiting examples of Factor VII variants having substantially the same biological activity as wild-type Factor VII include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189; and FVII variants exhibiting increased proteolytic stability as disclosed in WO 02/38162 (Scripps Research Institute); FVII variants having a modified Gla-domain and exhibiting an enhanced membrane binding as disclosed in WO 99/20767 (University of Minnesota); and FVII variants as disclosed in WO 01/58935 (Maxygen ApS).

Non-limiting examples of Factor VII variants having increased biological activity compared to wild-type FVIIa include FVII variants as disclosed in WO 01/83725, WO 02/22776, WO 02/077218, WO 03/27147, WO 03/37932; WO 02/38162 (Scripps Research Institute); and FVIIa variants with enhanced activity as disclosed in JP 2001061479 (Chemo-Sero-Therapeutic Res Inst.).

Non-limiting examples of Factor VII variants having substantially reduced or modified biological activity relative to wild-type Factor VII include R152E-FVIIa (Wildgoose et al., Biochem 29:3413-3420, 1990), S344A-FVIIa (Kazama et al., J. Biol. Chem. 270:66-72, 1995), FFR-FVIIa (Holst et al., Eur. J. Vasc. Endovasc. Surg. 15:515-520, 1998), and Factor VIIa lacking the Gla domain, (Nicolaisen et al., FEBS Letts. 317:245-249, 1993).

Examples of Factor VII polypeptides include, without limitation, wild-type Factor VII, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, Factor VIIa lacking the Gla domain; and P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys, and FVII having substitutions, deletions, or additions in the amino acid sequence Ile153-Arg223.

In some embodiments, the Factor VII polypeptide is human Factor VIIa (hFVIIa), preferably recombinantly made human Factor VIIa (rhVIIa).

In other embodiments, the Factor VII polypeptide is a Factor VII sequence variant.

In some embodiments, the Factor VII polypeptide has a glycosylation different from wild-type human Factor VII.

In various embodiments, e.g. those where the Factor VII polypeptide is a Factor VII-related polypeptide or a Factor VII sequence variant, the ratio between the activity of the Factor VII polypeptide and the activity of native human Factor VIIa (wild-type FVIIa) is at least about 1.25, preferably at least about 2.0, or 4.0, most preferred at least about 8.0, when tested in the “In Vitro Proteolysis Assay” (Assay 2) as described in the present specification.

In some embodiments, the Factor VII polypeptides are Factor VII-related polypeptides, in particular variants, wherein the ratio between the activity of said Factor VII polypeptide and the activity of native human Factor VIIa (wild-type FVIIa) is at least about 1.25 when tested in the “In Vitro Hydrolysis Assay” (see Assay 1 below); in other embodiments, the ratio is at least about 2.0; in further embodiments, the ratio is at least about 4.0.

Use of the Purified Bulk of the Factor VII Polypeptide

After collection of the fractions corresponding to the purified bulk of the Factor VII polypeptide, may be formulated into a solution, which may be dispensed into vials and freeze-dried. As an illustrative example of a final product corresponding to the commercially available, recombinantly-made FVII polypeptide composition NovoSeven® (Novo Nordisk A/S, Denmark), can be mentioned a a vial (1.2 mg) containing 1.2 mg recombinant human Factor VIIa, 5.84 mg NaCl, 2.94 mg CaCl₂, 2 H₂O, 2.64 mg GlyGly, 0.14 mg polysorbate 80, and 60.0 mg mannitol. This product is reconstituted to pH 5.5 by 2.0 mL water for injection (WFI) prior to use. When reconstituted, the protein solution is stable for use for 24 hours.

The overall manufacture of recombinant activated Factor VII (rFVIIa) is described by Jurlander, et al. in Seminars in Thrombosis and Hemostasis, Vol. 27, No. 4, 2001.

### EXAMPLES

Assays Suitable for Determining Biological Activity of Factor VII Polypeptides

Factor VII polypeptides useful in accordance with the present invention may be selected by suitable assays that can be performed as simple preliminary in vitro tests. Thus, the present specification discloses a simple test (entitled “In Vitro Hydrolysis Assay”) for the activity of Factor VII polypeptides.

In Vitro Hydrolysis Assay (Assay 1)

Native (wild-type) Factor VIIa and Factor VII polypeptide (both hereinafter referred to as “Factor VIIa”) may be assayed for specific activities. They may also be assayed in parallel to directly compare their specific activities. The assay is carried out in a microtiter plate (MaxiSorp, Nunc, Denmark). The chromogenic substrate D-Ile-Pro-Arg-p-nitroanilide (S-2288, Chromogenix, Sweden), final concentration 1 mM, is added to Factor VIIa (final concentration 100 nM) in 50 mM HEPES, pH 7.4, containing 0.1 M NaCl, 5 mM CaCl₂ and 1 mg/mL bovine serum albumin. The absorbance at 405 nm is measured continuously in a SpectraMax™ 340 plate reader (Molecular Devices, USA). The absorbance developed during a 20-minute incubation, after subtraction of the absorbance in a blank well containing no enzyme, is used for calculating the ratio between the activities of Factor VII polypeptide and wild-type Factor VIIa:

Ratio=(A405 nm Factor VII polypeptide)/(A405 nm Factor VIIa wild-type).

Based thereon, Factor VII polypeptides with an activity lower than, comparable to, or higher than native Factor VIIa may be identified, such as, for example, Factor VII polypeptides where the ratio between the activity of the Factor VII polypeptide and the activity of native Factor VII (wild-type FVII) is about 1.0 versus above 1.0.

The activity of the Factor VII polypeptides may also be measured using a physiological substrate such as Factor X (“In Vitro Proteolysis Assay”), suitably at a concentration of 100-1000 nM, where the Factor Xa generated is measured after the addition of a suitable chromogenic substrate (eg. S-2765). In addition, the activity assay may be run at physiological temperature.

### In Vitro Proteolysis Assay (Assay 2)

Native (wild-type) Factor VIIa and Factor VII polypeptide (both hereinafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 microM) in 100 μL 50 mM HEPES, pH 7.4, containing 0.1 M NaCl, 5 mM CaCl₂ and 1 mg/mL bovine serum albumin, are incubated for 15 min. Factor X cleavage is then stopped by the addition of 50 μL 50 mM HEPES, pH 7.4, containing 0.1 M NaCl, 20 mM EDTA and 1 mg/mL bovine serum albumin. The amount of Factor Xa generated is measured by the addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used for calculating the ratio between the proteolytic activities of Factor VII polypeptide and wild-type Factor VIIa:

Ratio=(A405 nm Factor VII polypeptide)/(A405 nm Factor VIIa wild-type).

Based thereon, Factor VII polypeptide with an activity lower than, comparable to, or higher than native Factor VIIa may be identified, such as, for example, Factor VII polypeptides where the ratio between the activity of the Factor VII polypeptide and the activity of native Factor VII (wild-type FVII) is about 1.0 versus above 1.0.

### Thrombin Generation Assay (Assay 3)

The ability of Factor VIIa or Factor VII polypeptides to generate thrombin can also be measured in an assay (Assay 3) comprising all relevant coagulation Factors and inhibitors at physiological concentrations (minus Factor VIII when mimicking hemophilia A conditions) and activated platelets (as described on p. 543 in Monroe et al. (1997) Brit. J. Haematol. 99, 542-547, which is hereby incorporated herein as reference).

### One-stage Coagulation Assay (Assay 4)

The biological activity of the Factor VII polypeptides may also be measured using a one-stage coagulation assay (Assay 4). For this purpose, the sample to be tested is diluted in 50 mM PIPES-buffer (pH 7.5), 0.1% BSA and 40 μl is incubated with 40 μl of Factor VII deficient plasma and 80 μl of human recombinant tissue factor containing 10 mM Ca2+ and synthetic phospholipids. Coagulation times are measured and compared to a standard curve using a reference standard in a parallel line assay.

### Preparation and Purification of Factor VII Polypeptides

Human purified Factor VIIa suitable for use in the present invention is preferably made by DNA recombinant technology, e.g. as described by Hagen et al., Proc. Natl. Acad. Sci. USA 83: 2412-2416, 1986, or as described in European Patent No. 0 200 421 (ZymoGenetics, Inc.).

Factor VII may also be produced by the methods described by Broze and Majerus, J. Biol. Chem. 255 (4): 1242-1247, 1980 and Hedner and Kisiel, J. Clin. Invest. 71: 1836-1841, 1983. These methods yield Factor VII without detectable amounts of other blood coagulation Factors. An even further purified Factor VII preparation may be obtained by including an additional gel filtration as the final purification step. Factor VII is then converted into activated Factor VIIa by known means, e.g. by several different plasma proteins, such as Factor XIIa, IX a or Xa. Alternatively, as described by Bjoern et al. (Research Disclosure, 269 September 1986, pp. 564-565), Factor VII may be completely activated by passing it through an ion-exchange chromatography column, such as Mono Q® (Pharmacia fine Chemicals) or the like, or by autoactivation in solution.

Factor VII-related polypeptides may be produced by modification of wild-type Factor VII or by recombinant technology. Factor VII-related polypeptides with altered amino acid sequence when compared to wild-type Factor VII may be produced by modifying the nucleic acid sequence encoding wild-type Factor VII either by altering the amino acid codons or by removal of some of the amino acid codons in the nucleic acid encoding the natural Factor VII by known means, e.g. by site-specific mutagenesis.

It will be apparent to those skilled in the art that substitutions can be made outside the regions critical to the function of the Factor VIIa molecule and still result in an active polypeptide. Amino acid residues essential to the activity of the Factor VII polypeptide, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for coagulant, respectively cross-linking activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The introduction of a mutation into the nucleic acid sequence to exchange one nucleotide for another nucleotide may be accomplished by site-directed mutagenesis using any of the methods known in the art. Particularly useful is the procedure that utilizes a super-coiled, double-stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with DpnI which is specific for methylated and hemi-methylated DNA to digest the parental DNA template and to select for mutation-containing synthesized DNA. Other procedures known in the art for creating, identifying and isolating variants may also be used, such as, for example, gene shuffling or phage display techniques.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J. C. Janson and Lars Ryden, editors, VCH Publishers, New York, 1989. Following purification, the preparation preferably contains less than 10% by weight, more preferably less than 5% and most preferably less than 1%, of non-Factor VII polypeptides derived from the host cell.

In the context of the present invention, the purification comprises at least one anion-exchange chromatography step.

If not completely activated by the process of the invention, Factor VII polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII polypeptides may be activated by passing it through an ion-exchange chromatography column, such as Mono Q® (Pharmacia) or the like, or by autoactivation in solution. The resulting activated Factor VII polypeptide may then be formulated and administered as described in the present application.

The following examples illustrate practice of the processes of the invention. These examples are included for illustrative purposes only and are not intended in any way to limit the scope of the invention claimed.

### Example 1

### Purification of a Bulk of Factor VII Polypeptide by Fractionated Elution From an Anion-Exchange Material with a Ca²⁺ Gradient Buffer

The process of the present invention can be conducted as follows:

A column comprising an anion-exchange material (Q-Sepharose FF from Amersham Biosciences) is washed with WFI (water for injection) and is subsequently equilibrated with a NaCl/Glygly buffer (175 mM NaCl and 10 mM Glygly).

A bulk of rFVII (recombinant Factor VII; M_{w} approx. 50,000) originating from a cell culture is applied to the column at pH 8.5 whereby essentially all of the Factor VII polypeptide bulk becomes bound to the column material. The content of Factor VII polypeptide with the desirable glycoform (sialylated glycoform) is around 78%. The load of the Factor VII polypeptide is approx. 20 g per litre of matrix (anion-exchange material in wet form).

The column is washed with a NaCl/Glygly buffer (175 mM NaCl and 10 mM Glygly) and subsequently with another NaCl/Glygly buffer (50 mM NaCl and 10 mM Glygly).

Fractionated elution is conducted with a gradient of CaCl₂ in a NaCl/Glygly buffer (0-15 mM CaCl₂; 50 mM NaCl and 10 mM Glygly) at a rate of 40 column volumes per hour.

A Ca²⁺ concentration threshold value, X, of 7.5 mM is set. Fractions corresponding to the Ca²⁺ concentration range of 7.5-13.5 mM are collected and are expected to have a content of Factor VII polypeptide with the desirable glycoform (sialylated glycoform) of above 90%.

1. An industrial-scale process for the purification of a bulk of a Factor VII polypeptide, said process comprising the step of:
(a) contacting the bulk of the Factor VII polypeptide with an anion-exchange material under conditions which facilitate binding of a portion of said bulk of the Factor VII polypeptide to said anion-exchange material;
(b) eluting said anion-exchange material with a first elution buffer comprising Ca²⁺ in a concentration of up to a threshold value of X mM; and
(c) eluting said anion-exchange material with a second elution buffer comprising Ca²⁺ in a concentration of more than the threshold value of X mM, and collecting a purified bulk of the Factor VII polypeptide as an eluate;
wherein the threshold value, X, is selected so that at least 5% by weight of the bulk of the Factor VII polypeptide is removed from said anion-exchange material in the first elution step (b), and at least 50% by weight of the bulk of the Factor VII polypeptide can be collected as the purified bulk of the Factor VII polypeptide in the second elution step (c).

2. The process according to claim 1, wherein the threshold value, X, is in the range of 3-12 mM.

3. An industrial-scale process for the purification of a bulk of a Factor VII polypeptide, said process comprising the step of:
(a) contacting the bulk of the Factor VII polypeptide with an anion-exchange material under conditions which facilitate binding of a portion of said bulk of the Factor VII polypeptide to said anion-exchange material;
(b) eluting said anion-exchange material with a first elution buffer comprising Ca²⁺ in a concentration of up to a threshold value of X mM; and
(c) eluting said anion-exchange material with a second elution buffer comprising Ca²⁺ in a concentration of more than the threshold value of X mM, and collecting a purified bulk of the Factor VII polypeptide as an eluate;
wherein the threshold value, X, is in the range of 3-12 mM.

4. The process according to claim 1, wherein the first elution step (b) is conducted by using a gradient buffer.

5. The process according to claim 4, wherein the gradient buffer has a final Ca²⁺ concentration corresponding to X.

6. The process according to claim 1, wherein the second elution step (c) is conducted by using a gradient buffer.

7. The process according to claim 6, wherein the gradient buffer has an initial Ca²⁺ concentration corresponding to just above X.

8. The process according to claim 1, wherein the first elution step (b) and the second elution step (c) are both conducted using a gradient buffer.

9. The process according to claim 8, wherein the initial concentration of Ca²⁺ of the gradient is in the range of 0-5 mM and a final concentration of Ca²⁺ of the gradient is in the range of 10-25 mM.

10. The process according to claim 1, wherein the first elution step (b) is conducted by using a first elution buffer in the form of a batch of a buffer with a (constant) Ca²⁺ concentration in the range of 1-7 mM, and the second elution step (c) is conducted by using a second elution buffer in the form of a batch of a buffer with a (constant) Ca²⁺ concentration in the range of 8-25 mM.

11. The process according to claim 1, wherein the anion-exchange material is a strong anion-exchange material.

12. An industrial scale process for the production and purification of a bulk of a Factor VII polypeptide, said process including the steps of
(i) producing a crude bulk of the Factor VII polypeptide in a cell culture, and
(ii) purifying said crude bulk of the Factor VII polypeptide by a purification sequence utilizing one or more anion-exchange purification processes,
wherein at least one of such anion-exchange purification processes is conducted as defined in claim 1.

13. The process according to claim 12, wherein the production step (i) is optimized with respect to the mass yield of the crude bulk of the Factor VII polypeptide.

14. The process according to claim 12, wherein the production of the crude bulk in the cell culture is conducted at a pH value of in the range of 7.0-7.6.
